(19) United States
(12) Patent Application Publication
(10) Pub. No.: US 2025/0064868 A1
(43) Pub. Date: Feb. 27, 2025

(54) USE OF A LIMOSILACTOBACILLUS FERMENTUM BACTERIAL STRAIN ALONE OR IN COMBINATION WITH SACCHAROMYCES CEREVISIAE FOR PREVENTING AND/OR TREATING VAGINAL CANDIDIASIS

(71) Applicant: LESAFFRE ET COMPAGNIE, PARIS (FR)

(72) Inventors: Nathalie BALLET, MARC EN BAROEUL (FR); Mickaël BOYER, MARCQ EN BAROEUL (FR); Luciana JIMENEZ CALDERON, LILLE (FR); Bertrand RODRIGUEZ, LAMBERSART (FR)

(73) Assignee: LESAFFRE ET COMPAGNIE, PARIS (FR)

(21) Appl. No.: 18/722,922

(22) PCT Filed: Dec. 20, 2022
(86) PCT No.: PCT/FR2022/052438

§ 371 Date: Jun. 21, 2024

(30) Foreign Application Priority Data
Dec. 21, 2021 (FR) FR2114188

(51) Int. Cl. A61K 35/742; A61K 36/064

(52) U.S. Cl. CPC A61K 35/742; A61K 36/064

(57) ABSTRACT

A Limosilactobacillus fermentum strain chosen among the strain deposited with the CNCM on Nov. 17, 2021, under number I-5777 or under number I-5778, for use thereof in the prevention and/or treatment of candidiasis vaginal. Also a Limosilactobacillus fermentum strain as defined above, characterized in that it is used in association with the Saccharomyces cerevisiae strain deposited with the CNCM on Oct. 17, 2007, under number I-3856, for use thereof in the prevention and/or treatment of vaginal candidiasis.

### FIELD

The present invention relates to the field of vaginal health. It concerns in particular the use of new Limosilactobacillus fermentum bacterial strains in the prevention and/or treatment of vaginal candidiasis. The invention also relates to the use of these new Limosilactobacillus fermentum bacterial strains in association with Saccharomyces cerevisiae in the prevention and/or treatment of vaginal candidiasis.

### BACKGROUND

Vaginal or vulvovaginal candidiasis (also called vaginal or vulvovaginal mycoses) are mainly caused by an infection by pathogenic fungi, namely Candida albicans yeast (hence the name candidiasis) in women's intimate areas. Candida albicans, naturally present in vaginal flora, can proliferate abnormally and lead to the development of vaginal candidiasis. Vaginal candidiasis or vaginal mycoses cause itching and inflammation of the vulva and vagina.

Vaginal mycoses are currently considered a major public health problem and constitute the second cause of vaginal infection after bacterial vaginitis. It is estimated that 75% of women develop a vaginal mycose at some point. Around 50% of them experience at least 2 to 3 episodes of vaginal mycoses during their life. 10 to 20% of women suffer from recurrent vaginal mycoses with an average of 4 episodes per year.

The antimycotic treatments usually used currently are based on employing chemical compounds, mainly azole derivatives such as, for example, fluconazole, ketoconazole, econazole, miconazole, or clotrimazole, generally administered in the form of a cream or suppository to be inserted into the vagina. In some cases, oral treatment supplements the local treatment.

However, one of the major problems with this type of treatment is that, when there are repeated vaginal mycoses, the pathogenic fungus, in particular Candida albicans, can develop resistance to existing antimycotic treatments.

Another problem with the use of azole derivatives for treating mycoses is the feeling of irritation and heat in the vulval area.

In order to offer alternatives to treatments based on azole derivatives, probiotics have been described in the literature.

In 2001, probiotics were defined by the World Health Organization (WHO) as “live microorganisms which, when administered in adequate amounts, confer a health benefit beyond the effects of traditional nutritional products.”

Document PCT/SE2006/001311 thus describes the use of a strain of Lactobacillus fermentum, “Ess-1”, deposited with the “Deutsche Sammlung von Mikroagencen und Zellkulturen GmbH” under number “DSM17851”, in the prevention and/or or treatment of vaginal candidiasis.

Document PCT/FR2013/051643 describes the use of a Saccharomyces cerevisiae strain deposited with the CNCM under number I-3856, in the prevention and/or treatment of vaginal mycoses, and in particular vaginal candidiasis.

Probiotics do not have the abovementioned disadvantages of chemical treatments (resistance phenomenon, side effects such as irritation, burning) while providing an effectiveness that is at least comparable to said treatments. Probiotics thus represent natural alternative solutions to chemical compounds, without the adverse effects.

### SUMMARY

One of the aims of the present invention is therefore to propose new probiotics for preventing and/or treating vaginal candidiasis.

The inventors have surprisingly found that the use of at least one Limosilactobacillus fermentum bacterial strain deposited on Nov. 17, 2021 in the CNCM (National Collection of Microorganism Cultures, 25, rue du Docteur Roux, 75724 Paris cedex 15, France) under number I-5777 or under number I-5778 allowed achieving this objective.

One object of the invention thus resides in a Limosilactobacillus fermentum strain chosen among the strain deposited with the CNCM on Nov. 17, 2021 under number I-5777 or under number I-5778.

Bear in mind that the probiotic effect of a given strain, whether it is a strain of bacteria or a strain of yeast, is specific to this strain, and not to the genus and species considered.

Another object of the invention resides in a Limosilactobacillus fermentum strain chosen among the strain deposited with the CNCM on Nov. 17, 2021 under number I-5777 or under number I-5778, for its use in the prevention and/or treatment of vaginal candidiasis.

The Limosilactobacillus fermentum strain is a strain of bacteria, or bacterial strain. The term “bacterial strain” refers to a relatively homogeneous population of bacterial cells.

A bacterial strain is obtained from the isolation of a clone, a clone being a population of cells obtained from a single bacterial cell.

The present invention also relates to the bacteria obtained by culturing the Limosilactobacillus fermentum strain chosen among the strain deposited with the CNCM on Nov. 17, 2021 under number I-5777 or under number I-5778, for its use in the prevention and/or treatment of vaginal candidiasis.

In the present application, the term “bacteria” is used in the plural but may also be used in the singular. “Bacteria” thus designates the bacteria or bacterium obtained by culturing the Limosilactobacillus fermentum strain as defined above. The phrases “bacteria obtained by culturing the Limosilactobacillus fermentum strain deposited with the CNCM on Nov. 17, 2021 under number I-5777”, “bacteria obtained by culturing strain I-5777”, and “bacteria I-5777” will be used interchangeably in the present application, these three phrases having the same meaning as the one given above for the term “bacteria”.

Similarly, the phrases “bacteria obtained by culturing the Limosilactobacillus fermentum strain deposited with the CNCM on Nov. 17, 2021 under number I-5778”, “bacteria obtained by culturing strain I-5778”, and “bacteria I-5778” will be used interchangeably in the present application, these three phrases having the same meaning as given above.

For the purposes of the present application, the terms “bacterial strain”, “strain of bacteria”, “Limosilactobacillus fermentum strain” all designate strain I-5777 and/or strain I-5778.

For the purposes of the present application, the Limosilactobacillus fermentum bacteria of the invention designates bacteria I-5777 and/or bacteria I-5778.

As an indication, the Limosilactobacillus fermentum bacteria of the invention may be obtained by culturing strain I-5777 or strain I-5778 overnight at 37° C., without stirring, in a MRS (“De Man Rogosa Sharpe”) agar culture medium (Biomerieux-AEB140652) at a pH of 6.5.

The Limosilactobacillus fermentum bacteria may, however, also be obtained by an industrial-scale process which comprises the following steps:
cultivating the bacterial strain in an optimal medium for biomass production,
centrifuging to separate the bacteria thus produced from its culture medium, in order to obtain a concentrated biomass in cream containing 10 to 20% dry matter, said concentrated biomass being called the “centrifugate”,
freezing the centrifugate, preferably with liquid nitrogen,
possibly storing the frozen centrifugate at −80° C. for a period of up to one year,
freeze-drying the frozen centrifugate to obtain a powder containing 90 to 98% dry matter.

The powder obtained by freeze-drying corresponds to what is meant in the present application by “dry form, preferably freeze-dried”.

According to one embodiment of the invention, the bacteria as defined above, for its use in the prevention and/or treatment of vaginal candidiasis, is presented in frozen form or in dry form, preferably in dry form, and more preferably still in freeze-dried dry form.

According to one advantageous embodiment of the invention, bacterial strain or bacteria I-5777 is used, for its use in the prevention and/or treatment of vaginal candidiasis, in dry form, preferably freeze-dried, at a daily dose of 11×10⁷ to 1×10¹⁰ CFU, preferably 2×10⁸ to 2×10⁹ CFU.

According to yet another advantageous embodiment of the invention, bacterial strain or bacteria I-5778 is used, in dry form, preferably freeze-dried, for its use in the prevention and/or treatment of vaginal candidiasis, at a daily dose of 1×10⁷ to 1×10¹⁰ CFU, preferably 2×10⁸ to 2×10⁹ CFU. The acronym “CFU” stands for “Colony Forming Unit”.

The term “vaginal” is used broadly in the present application, and the terms “vaginal” and “vulvovaginal” are synonymous here.

“Vaginal candidiasis” is understood to mean a particular type of vaginal mycose due to fungi of the genus Candida, for example such as those chosen from the group comprising Candida albicans, Candida tropicalis, Candida pseudotropicalis, Candida krusei, Candida glabrata, and mixtures thereof.
Vaginal candidiasis is also called Candidal vaginitis.

According to another embodiment, an object of the invention is a Limosilactobacillus fermentum strain as defined above or a Limosilactobacillus fermentum bacteria as defined above, for its use in the prevention and/or treatment of vaginal candidiasis due to fungi chosen from the group comprising Candida albicans, Candida tropicalis, Candida pseudotropicalis, Candida krusei, Candida glabrata, and mixtures thereof.

The Limosilactobacillus fermentum strain or the Limosilactobacillus fermentum bacteria may be used preventively in women with predispositions and/or sensitivity to vaginal candidiasis, or may be used curatively, for example during infectious episodes.

The present inventors also discovered that when one of these two strains or both strains of bacteria were associated with the Saccharomyces cerevisiae strain deposited on Oct. 17, 2007 with the CNCM (Collection Nationale de Cultures de Microorganisms, 25, rue du Docteur Roux, 75724 Paris cedex 15, France) under number I-3856, then a particularly surprising synergistic effect is obtained, leading to particularly effective means for treating vaginal candidiasis.

In this regard, another object of the invention resides in a Limosilactobacillus fermentum bacterial strain or bacteria as defined above, for use in the prevention and/or treatment of vaginal candidiasis, characterized in that it is used in association with the Saccharomyces cerevisiae strain deposited with the CNCM on Oct. 17, 2017 under number I-3856 and/or with the yeast obtained by culturing said Saccharomyces cerevisiae strain.

The Saccharomyces cerevisiae strain is a yeast strain. The term “yeast strain” refers to a relatively homogeneous population of yeast cells. A yeast strain is obtained from the isolation of a clone, a clone being a population of cells obtained from a single yeast cell.

For the purposes of the present application, Saccharomyces cerevisiae yeast designates yeast I-3856. Thus, according to one embodiment of the invention, the Limosilactobacillus fermentum bacterial strain or bacteria may be associated with the Saccharomyces cerevisiae strain and/or with the Saccharomyces cerevisiae yeast.

In other words, according to one advantageous embodiment of the invention, Limosilactobacillus fermentum bacteria I-5777 and/or I-5778 is (are) associated with Saccharomyces cerevisiae yeast I-3856.

As an indication, the Saccharomyces cerevisiae yeast may be obtained by culturing strain number I-3856 in a culture medium, for example as described in the reference book “Yeast Technology”, 2nd edition, 1991, G. Reed and T. W. Nagodawithana, published by Van Nostrand Reinhold, ISBN 0-442-31892-8.

However, Sc yeast I-3856 may also be obtained by an industrial-scale process which comprises the following steps:
cultivating a yeast strain in a culture medium in several stages, first semi-anaerobic, then aerobic,
centrifuging the yeast thus produced to separate it from its culture medium in order to obtain a liquid yeast cream containing 12 to 25% dry matter, or even a higher amount of dry matter, in particular if the yeast cream is mixed with osmolytic products.

The terms “yeast obtained by cultivating the Saccharomyces cerevisiae strain deposited with the CNCM under number I-3856”, “yeast obtained by cultivating strain I-3856”, and “yeast Sc I-3856” will be used interchangeably in this application, these three terms having the same meaning as given above.

The Saccharomyces cerevisiae strain deposited with the CNCM under number I-3856 may also be designated as “strain Sc I-3856”.

According to yet another advantageous embodiment of the invention, yeast Sc I-3856, used in association with the Limosilactobacillus fermentum bacterial strain or bacteria, is alive. The live yeast is then in the form of dry yeast or fresh yeast, preferably in the form of dry yeast.

For example, in the case of the association of yeast Sc I-3856 with the Limosilactobacillus fermentum bacterial strain or bacteria, for preventing and/or treating vaginal candidiasis:
the Limosilactobacillus fermentum bacterial strain or bacteria in dry form, preferably freeze-dried, is used at a daily dose of 1×10⁷ to 1×10¹⁰ CFU, preferably 2×10⁸ to 2×10⁹ CFU, and
the yeast Sc I-3856, in dry form, is used at a daily dose of 1×10⁷ to 1×10¹¹ CFU, preferably 1×10⁹ to 1×10¹⁰ CFU, and even more preferably 2.5×10⁹ to 5×10⁹ CFU.

Fresh yeast is characterized by a high water content. Fresh yeast is for example chosen among liquid yeast or pressed yeast.

Liquid yeast, also called “liquid cream yeast”, is an aqueous suspension comprising from 12% to 50% yeast dry matter, more generally from 12% to 25% yeast dry matter.

Pressed yeast is obtained by a/filtering a liquid cream yeast, generally on a rotary drum vacuum filter, to obtain fresh dehydrated yeast containing 26% to 37% dry matter, and b/kneading said fresh dehydrated yeast, and c/extrusion.

Pressed yeast contains 26% to 37% yeast dry matter. Pressed yeast may or may not be crumbled. Among the dry yeasts, we can cite for example active dry yeast, instant dry yeast, and frozen intermediate moisture yeast.

An advantage of dry yeast is its long shelf life.

An active dry yeast or an instant dry yeast has a yeast dry matter content that is greater than 90%, preferably a dry matter content of between 92% to 96%.

Active dry yeast is obtained by dehydration of pressed or liquid yeast, via the joint action of heat (at low temperature) and mechanical activity, which allows transforming a paste product (pressed or liquid yeast) into a dry product in the form of spherules. For example, active dry yeast is obtained by extrusion and drying in a fluidized bed a pressed yeast or liquid yeast.

Active dry yeast is in the form of spherules of a diameter that is generally between 0.1 μm and 2.5 mm.

Instant dry yeast is obtained by dehydration of pressed or liquid yeast via the action of a hot air gradient which transforms a pasty product (pressed or liquid yeast) into thin dry vermicelli. To remain stable, instant dry yeast must then be packaged in the absence of oxygen.

Frozen intermediate moisture yeast comprises, for example, 70% to 85% yeast dry matter.

According to yet another advantageous embodiment of the invention, yeast Sc I-3856, used in association with the Limosilactobacillus fermentum bacterial strain or bacteria in the prevention and/or treatment of vaginal mycoses, is in the form of active dry yeast or in the form of instant dry yeast.

According to yet another advantageous embodiment of the invention, yeast Sc I-3856, used in association with the Limosilactobacillus fermentum bacterial strain or bacteria, in the prevention and/or treatment of vaginal mycoses, is presented in the form of inactive yeast, also called deactivated yeast.

Inactive yeast is yeast in which metabolism has been irreversibly stopped.

The inactive yeast may be obtained by techniques well known to those skilled in the art, such as heat treatment of the yeast, treatment consisting of subjecting the yeast to several successive freeze-thaw cycles, spray treatment, atomization treatment, or a combination of these treatments.

For example, in the case of the association of inactive yeast with the Limosilactobacillus fermentum bacterial strain or bacteria for the prevention and/or treatment of vaginal candidiasis:
the Limosilactobacillus fermentum bacterial strain or bacteria, in dry form, preferably freeze-dried, is used at a daily dose of 1×10⁷ to 1×10¹⁰ CFU, preferably 2×10⁸ to 2×10⁹ CFU, and
yeast Sc I-3856, in the form of inactive yeast, is used at a daily dose of 1 to 10,000 mg, and preferably of 100 to 1000 mg.

According to yet another advantageous embodiment of the invention, yeast Sc I-3856, used in association with the Limosilactobacillus fermentum bacterial strain or bacteria, is used in the form of a yeast derivative, said derivative being chosen among the cell wall of said yeast, the cell wall glucans of said yeast, the cell wall mannoproteins of said yeast, or mixtures thereof.

Cell wall of yeast refers broadly to both the wall and the plasma membrane of yeast. Conventionally, the cell wall of yeast is obtained by a process comprising a step of autolysis of the yeast followed by a step of separating the soluble fraction from the insoluble fraction, the isolated insoluble fraction corresponding to the yeast wall which may then be dried.

For example, in the case of the association of a cell wall of yeast Sc I-3856 with the Limosilactobacillus fermentum bacterial strain or bacteria, for the prevention and or treatment of vaginal candidiasis:
the Limosilactobacillus fermentum bacterial strain or bacteria, in dry form, preferably freeze-dried, is used at a daily dose of 1×10⁷ to 1×10¹⁰ CFU, preferably 2×10⁸ to 2×10⁹ CFU, and
the cell wall of yeast Sc I-3856 is used at a daily dose of 0.5 to 5,000 mg, preferably 50 to 500 mg. For example, in the case of combining cell wall glucans from yeast Sc I-3856 with the Limosilactobacillus fermentum bacterial strain or bacteria, for the prevention and/or treatment of vaginal candidiasis:
the Limosilactobacillus fermentum bacterial strain or bacteria, in dry form, preferably freeze-dried, is used at a daily dose of 1×10⁷ to 1×10¹⁰ CFU, preferably 2×10⁸ to 2×10⁹ CFU, and
cell wall glucans of yeast Sc I-3856 are used at a daily dose of 0.125 to 1250 mg, and preferably 12.5 to 125 mg.

Again as an example, in the case of the association of cell wall mannoproteins of yeast Sc I-3856 with the Limosilactobacillus fermentum bacterial strain or bacteria, for the prevention and/or treatment of vaginal candidiasis:
the Limosilactobacillus fermentum bacterial strain or bacteria, in dry form, preferably freeze-dried, is used at a daily dose of 1×10⁷ to 1×10¹⁰ CFU, preferably 2×10⁸ to 2×10⁹ CFU, and
cell wall mannoproteins of yeast Sc I-3856 are used at a daily dose of 0.125 to 1250 mg, and preferably 12.5 to 125 mg.

According to an advantageous embodiment of the invention, the Limosilactobacillus fermentum bacterial strain and/or bacteria on the one hand and strain Sc I-3856 and/or yeast Sc I-3856 on the other hand, are administered simultaneously or sequentially, and preferably simultaneously.

When the administration is sequential, yeast Sc I-3856 could for example be administered in the morning and bacteria I-5777 and/or I-5778 in the evening.

Thus, the invention also relates to the Limosilactobacillus fermentum bacterial strain and/or bacteria, in association with strain Sc I-3856 and/or yeast Sc I-3856, for simultaneous or sequential administration, for use in the prevention and/or treatment of vaginal candidiasis.

Depending on the desired mode of administration. (simultaneous or sequential), the Limosilactobacillus fermentum bacterial strain and/or bacteria on the one hand, and strain Sc I-3856 and/or yeast Sc I-3856 on the other hand, are in the form of a single composition or in the form of a separate composition.

A single composition means that the Limosilactobacillus fermentum bacterial strain and/or bacteria on the one hand and strain Sc I-3856 and/or yeast Sc I-3856 on the other hand are found within a single composition.

A “separate” composition means that the Limosilactobacillus fermentum bacterial strain and/or bacteria is found in one composition and strain Sc I-3856 and/or yeast Sc I-3856 is found in another composition that is separate from the composition comprising the bacterial strain and/or bacteria.

According to a preferred embodiment, the invention relates to bacterial strain and/or bacteria I-5777, in association with strain Sc I-3856 and/or yeast Sc I-3856, for use in the prevention and/or treatment of vaginal candidiasis.

Preferably bacterial strain and/or bacteria I-5777 on the one hand, and strain Sc I-3856 and/or yeast Sc I-3856 on the other hand, are present within a single composition and are therefore administered simultaneously.

According to another preferred embodiment, the invention relates to bacterial strain and/or bacteria I-5778, in association with strain Sc I-3856 and/or yeast Sc I-3856, for use in the prevention and/or treatment of vaginal candidiasis.

Preferably bacterial strain and/or bacteria I-5778 on the one hand and strain Sc I-3856 and/or yeast Sc I-3856 on the other hand, are present within a single composition and are therefore administered simultaneously.

Yet another object of the invention relates to a composition comprising:
a Limosilactobacillus fermentum strain chosen among the strain deposited with the CNCM on Nov. 17, 2021 under number I-5777 or under number I-5778, and/or a bacteria obtained by culturing said Limosilactobacillus fermentum strain,
possibly an excipient,
   for its use in the prevention and/or treatment of vaginal candidiasis.

Another object of the invention resides in a composition comprising:
a Limosilactobacillus fermentum strain chosen among the strain deposited with the CNCM on Nov. 17, 2021 under number I-5777 or under number I-5778, and/or a bacteria obtained by culturing said Limosilactobacillus fermentum strain,
the Saccharomyces cerevisiae strain deposited with the CNCM on Oct. 17, 2007 under number I-3856 and/or the yeast obtained by culturing said Saccharomyces cerevisiae strain,
possibly an excipient,
   for its use in the prevention and/or treatment of vaginal candidiasis.

The composition according to the invention for a use as defined above may be a pharmaceutical composition, a food supplement, or a food composition.

Food composition refers to any food, drink, or confectionery. Examples of a food composition are a granola bar, a chewing gum, a dairy product.

When the composition is a food composition, then some examples of an excipient are lactose, corn starch, carboxymethylcellulose, mannitol, sucrose.

“Food supplement” is understood to mean a food whose purpose is to supplement the normal diet and which constitutes a concentrated source of nutrients or other substances having a nutritional or physiological effect, alone or in combination.

A food supplement is marketed in dose form, for example in forms such as a capsule, lozenge, tablet, pill, and other similar forms, a sachet of powder, ampoule of liquid, bottle fitted with a dropper, and other similar forms of liquid or powder preparations intended to be taken in small measured units.

The food composition and the food supplement are intended for oral administration.

The pharmaceutical composition of the invention is intended for oral or vaginal administration, preferably oral. The excipients used in the pharmaceutical composition are excipients which are conventionally used and which are suitable for the preparation of oral or vaginal forms.

Examples of a pharmaceutical composition in a form suitable for oral administration include a tablet, a capsule, a gelatin capsule, a sachet, a powder, a cream, a syrup, or an ampoule.

Examples of a pharmaceutical composition in a form suitable for vaginal administration include a suppository, a capsule, a gelatin capsule, a cream, or a tablet.

The daily dosage depends on both the method of administration (oral or vaginal) and the type of treatment (curative or preventive).

The composition for a use as defined above is in a form suitable for oral or vaginal administration, preferably oral.

According to one advantageous embodiment of the invention, the composition for a use as defined above does not comprise any active ingredients other than Limosilactobacillus fermentum strain I-5777 and/or I-5778 and/or Limosilactobacillus fermentum bacteria I-5777 and/or I-5778 on the one hand, and strain Sc I-3856 and/or yeast Sc I-3856 on the other hand.

Thus, the present invention also relates to a composition for a use as defined above, characterized in that it comprises or in that it is composed of:
Limosilactobacillus fermentum strain I-5777 and/or I-5778, and/or Limosilactobacillus fermentum bacteria I-5777 and/or I-5778 on the one hand,
strain Sc I-3856 and/or yeast Sc I-3856 on the other hand,
possibly an excipient.

If the composition comprises an excipient, then the excipient will be chosen according to the end use of the composition (pharmaceutical, food, food supplement).

According to another advantageous embodiment of the invention, the composition for a use as defined above is characterized in that it further comprises an active ingredient, in particular chosen from the group comprising vitamin K, vitamin B9, glutathione, S-adenosyl-L-methionine (SAM-e), chondroitin sulfate, and mixtures thereof. Such a composition is preferably intended for oral administration.

According to yet another embodiment of the invention, the composition, for a use as defined above, is more particularly characterized in that:
Limosilactobacillus fermentum bacterial strain or bacteria I-5777 and/or I-5778 is in dry form, preferably freeze-dried, and is present in a quantity ranging from 1×10⁷ to 1×10¹⁰ CFU, preferably from 2×10⁸ to 2×10⁹ CFU, and
yeast Sc I-3856 is in dry form, and is present in a quantity ranging from 1×10⁷ to 1×10¹¹ CFU, preferably from 1×10⁹ to 1×10¹⁰ CFU, and more preferably still from 2.5×10⁹ to 5×10⁹ CFU.

According to another embodiment, the composition, for a use as defined above, is more particularly characterized in that:
Limosilactobacillus fermentum bacterial strain or bacteria I-5777 and/or I-5778 is in dry form, preferably freeze-dried, and is present in a quantity ranging from 1×10⁷ to 1×10¹⁰ CFU, preferably from 2×10⁸ to 2×10⁹ CFU, and
yeast Sc I-3856 is in the form of inactive yeast, and is present in a quantity ranging from 1 to 10,000 mg, and preferably from 100 to 1000 mg.

According to yet another embodiment, the composition, for a use as defined above, is more particularly characterized in that:
Limosilactobacillus fermentum bacterial strain or bacteria I-5777 and/or I-5778 is in dry form, preferably freeze-dried, and is present in a quantity ranging from 1×10⁷ to 1×10¹⁰ CFU, preferably from 2×10⁸ to 2×10⁹ CFU, and
yeast Sc I-3856 is in the form of yeast cell walls, and is present in a quantity ranging from 0.5 to 5,000 mg, preferably from 50 to 500 mg.

According to another embodiment, the composition, for a use as defined above, is more particularly characterized in that:
Limosilactobacillus fermentum bacterial strain or bacteria I-5777 and/or I-5778 is in dry form, preferably freeze-dried, and is present in a quantity ranging from 1×10⁷ to 1×10¹⁰ CFU, preferably from 2×10⁸ to 2×10⁹ CFU, and
yeast Sc I-3856 is in the form of cell wall glucans and is present in a quantity ranging from 0.125 to 1250 mg, preferably from 12.5 to 125 mg.

According to another embodiment, the composition, for a use as defined above, is more particularly characterized in that:
Limosilactobacillus fermentum bacterial strain or bacteria I-5777 and/or I-5778 is in dry form, preferably freeze-dried, and is present in a quantity ranging from 1×10⁷ to 1×10¹⁰ CFU, preferably from 2×10⁸ to 2×10⁹ CFU, and
yeast Sc I-3856 is in the form of cell wall mannoproteins and is present in a quantity ranging from 0.125 to 1250 mg, preferably from 12.5 to 125 mg.

### BRIEF DESCRIPTION OF DRAWINGS

Other features, details and advantages will become apparent upon reading the detailed description below, and upon analyzing the appended drawings.

FIG. 1 is a histogram resulting from a first experiment and which shows the percentage of inhibition of the formation of Candida albicans hyphae by lactobacilli denoted “LS1” to “LS14”, lactobacilli LS4 and LS5 corresponding to the bacterial strains of the invention, namely strains CNCM I-5777 and CNCM I-5778 respectively.

The y-axis represents the percentage of hyphal formation normalized to Candida albicans.

The “CA” group (Candida albicans) represents the control group.

Significant differences compared to the control group are annotated with asterisks ** p<0.01.

FIG. 2 is a histogram resulting from a second experiment and which shows the percentage of inhibition of the formation of Candida albicans hyphae, respectively by the control CA and by lactobacilli LS4 (CNCM I-5777), LS5 (CNCM I-5778), and LS7.

The y-axis represents the percentage of hyphal formation normalized to Candida albicans.

Significant differences compared to the control group are annotated with asterisks **** p<0.0001.

FIG. 3 is a histogram resulting from a third experiment and which shows the percentage of inhibition of the formation of Candida albicans hyphae, respectively by the control CA and by lactobacilli LS4 (CNCM I-5777) and LS5 (CNCM I-5778).

The y-axis represents the percentage of hyphal formation normalized to Candida albicans.

Significant differences compared to the control group are annotated with asterisks **** p<0.0001.

FIG. 4 is a histogram which shows the percentage of inhibition of the formation of Candida albicans hyphae, respectively by the control CA, alone or combined with Saccharomyces cerevisiae CNCM I-3856 (“Sc”), and by lactobacilli (LS4, LS7 and LS11) alone (light gray histograms) or combined with Saccharomyces cerevisiae CNCM I-3856 (Sc) (histograms with hatching).

The y-axis represents the percentage of hyphal formation normalized to Candida albicans. “−Sc” indicates the absence of Saccharomyces cerevisiae, while “+Sc” indicates the presence of Saccharomyces cerevisiae.

Significant differences compared to the control group are annotated with asterisks **** p<0.0001.

### DETAILED DESCRIPTION

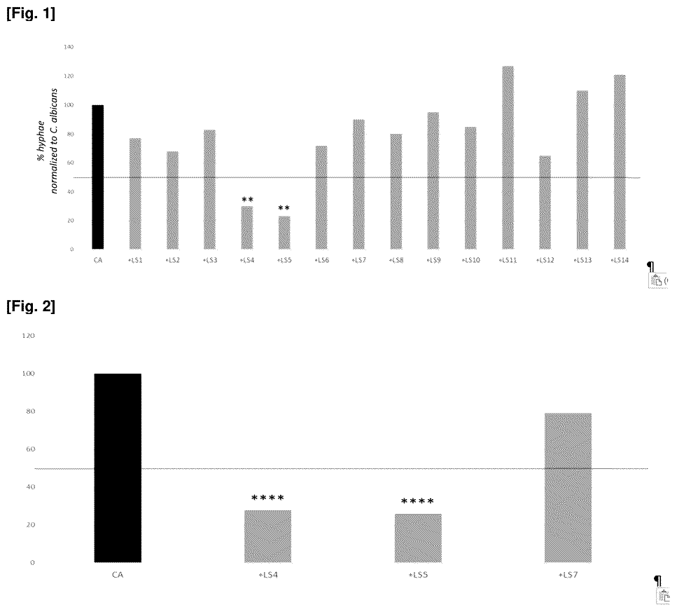

In Example 1 below, reference is made to FIGS. 1 to 4.

### Example 1

### Hyphae Test

The hyphae test, carried out under in vitro conditions, allows evaluating the capacity of strains (of bacteria or yeast) to block the formation of Candida albicans hyphae (i.e. to inhibit the formation of hyphae).

Indeed, the formation of hyphae is a crucial step in the virulence and invasion capacity of Candida albicans: hyphal morphogenesis is thus an important virulence factor of C. albicans.

Hyphal inhibition/reduction represents an interesting approach to combating Candida infections.

### Strains Tested

Fourteen strains of lactobacilli were tested (LS1 to LS14), alone or in association with S. cerevisiae strain CNCM I-3856.

Strains LS1 to LS14 are strains isolated from sourdough starter.
LS1: Lactiplantibacillus plantarum, identification reference BCC_002300,
LS2: Lactiplantibacillus plantarum, identification reference BCC_002377,
LS3: Lactiplantibacillus plantarum, identification reference BCC_003240,
LS4: Limosilactobacillus fermentum, identification reference BCC_002284 (strain deposited with the
CNCM on Nov. 17, 2021 under number I-5777),
LS5: Limosilactobacillus fermentum, identification reference BCC_002321 (strain deposited with the
CNCM on Nov. 17, 2021 under number I-5778),
LS6: Lacticaseibacillus paracasei, identification reference BCC_002365,
LS7: Lactiplantibacillus plantarum, identification reference BCC_002378 (strain deposited with the
CNCM on Nov. 17, 2021 under number I-5779),
LS8: Lactiplantibacillus plantarum, identification reference BCC_004104,
LS9: Lactiplantibacillus plantarum, identification reference BCC_004132,
LS10: Lactiplantibacillus plantarum, identification reference BCC_004299,
LS11: Lacticaseibacillus paracasei, identification reference BCC_002274,
LS12: Lacticaseibacillus paracasei, identification reference BCC_002301,
LS13: Lacticaseibacillus paracasei, identification reference BCC_003027,
LS14: Lacticaseibacillus paracasei, identification reference BCC_003258.

### Test Protocol

Protocol with Lactobacilli Alone

The concentrations of overnight cultures of lactobacilli strains (LS1 to LS14) and of C. albicans strains are estimated by spectrophotometry (optical density at 600 nm). The cultures are then adjusted to a concentration of 1×10⁹ CFU/ml for lactobacilli and 1×10⁶ CFU/ml for C. albicans.

Mixtures of C. albicans with lactobacilli are created by adding 50 μl of suspension of each test microorganism to 125 μl of fetal calf serum (FCS), supplemented with YPD broth (yeast-peptone-dextrose broth) for a total volume of 500 μl.

For each condition, four technical repetitions are included (the results come from four different wells but from the same initial overnight culture). After 3 hours of incubation at 37° C., 2 μl of the mixtures are used for a microscopic evaluation by counting the number of yeast cells and the number of hypha-forming cells. At least 100 cells are counted and ratios are calculated and normalized to the negative control (i.e. C. albicans+FCS).

Protocol with Lactobacilli in Combination with Saccharomyces cerevisiae

The supernatant from the overnight culture of S. cerevisiae (Sc I-3856) is obtained by centrifugation (10 min, 2000 g) and filtration (0.20 μm cellulose acetate filter).

The concentrations of overnight cultures of lactobacilli and C. albicans strains are estimated by spectrophotometry (optical density at 600 nm). The cultures are then adjusted to a concentration of 1×10⁹ CFU/ml for lactobacilli and 1×10⁶ CFU/ml for C. albicans.

Mixtures of C. albicans with lactobacilli and the supernatant of S. cerevisiae are created by adding 50 μl of suspension of each test microorganism to 125 μl of fetal calf serum (FCS), supplemented with YPD broth for a total volume of 500 μl.

The rest of the protocol is identical to the protocol described above for lactobacilli alone.

### Results

The results obtained are commented on below and are illustrated in FIGS. 1 to 4. Note, in each of these figures, the zero percent (0%) inhibition of hyphal formation with the control CA (C. albicans) (which means 100% formation of C. albicans hyphae).

### Lactobacilli Alone (FIGS. 1 to 3)

It is apparent from FIG. 1 that, among the 14 lactobacilli tested (LS1 to LS14), only Limosilactobacillus fermentum strains LS4 (CNCM I-5777) and LS5 (CNCM I-5778) provide strong inhibition of the formation of C. albicans hyphae.

Indeed, with strains LS4 and LS5, approximately 72% inhibition of hyphal formation (i.e. approximately 28% hyphal formation) (LS4) and approximately 78% inhibition of hyphal formation of C. albicans hyphae (i.e. approximately 22% hyphal formation) (LS5) is observed respectively.

In FIG. 2, strains LS4 (CNCM I-5777) and LS5 (CNCM I-5778) were compared to strain LS7 (CNCM I-5779) because this strain is representative of a large number of the strains tested. It is also apparent from FIG. 2 that strains LS4 (CNCM I-5777) and LS5 (CNCM I-5778) provide strong inhibition of the formation of C. albicans hyphae in comparison to strain LS7 (CNCM I-5779). Indeed, with strains LS4 and LS5 we respectively observe approximately 74% inhibition (LS4) and approximately 75% inhibition (LS5), while with strain LS7 (CNCM I-5779) the inhibition is barely 20%.

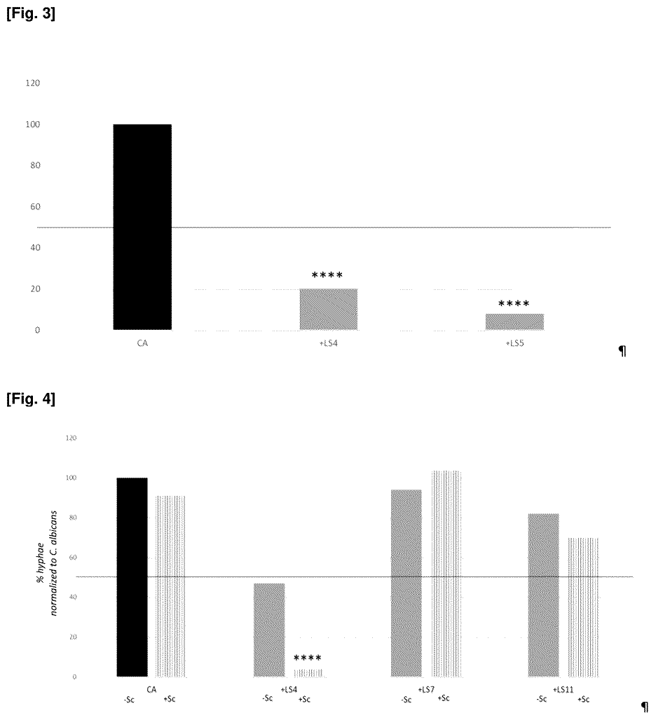

In FIG. 3, only strains LS4 (CNCM I-5777) and LS5 (CNCM I-5778) were compared. Once again, the results obtained are promising, since these strains result in strong inhibition of the formation of C. albicans hyphae, namely approximately 81% (LS4) and 93% (LS5) inhibition, respectively, of the formation of C. albicans hyphae.

Lactobacilli in Combination with Saccharomyces cerevisiae (FIG. 4)

It is apparent from FIG. 4 that the association of LS4 (CNCM I-5777) with S. cerevisiae CNCM I-3856 leads to an unexpected synergistic effect, which is not the case in the respective association of strains LS7 (CNCM I-5779) and LS11 with Sc I-3856.

Indeed, the use of S. cerevisiae strain CNCM I-3856 (see “CA+Sc”) alone (i.e. not associated with a lactobacillus) leads to inhibition of the formation of C. albicans hyphae by approximately 9% under the experimental conditions involved. The use of strain LS4 alone (see “LS4−Sc”) leads to inhibition of the formation of C. albicans hyphae by approximately 52%. However, when strains LS4 and Sc CNCM I-3856 are combined, then the inhibition of the formation of C. albicans hyphae is almost total inhibition since it is approximately 97%.

The present invention is not limited to the examples described above solely by way of example, but encompasses all variants conceivable to those skilled in the art within the context of the protection sought.

### Reference to Biological Material Deposited in Libraries

In the present application, reference is made to the following biological materials and to the derived variant or mutant strains:
identification reference “BCC_002284” and registration number “I-5777” deposited with the National Collection of Cultures of Microorganisms (CNCM) (25, rue du Docteur Roux, 75724 Paris cedex 15) in France, on 17 Nov. 2021 by Lesaffre et Compagnie whose address is 41 rue Etienne Marcel, 75009 Paris;
identification reference “BCC_002321” and registration number “I-5778” deposited with the National Collection of Cultures of Microorganisms (CNCM) (25, rue du Docteur Roux, 75724 Paris cedex 15) in France, on 17 Nov. 2021 by Lesaffre et Compagnie whose address is 41 rue Etienne Marcel, 75009 Paris;
identification reference “BCC_002378” and registration number “I-5779” deposited with the National Collection of Cultures of Microorganisms (CNCM) (25, rue du Docteur Roux, 75724 Paris cedex 15) in France, on 17 Nov. 2021 by Lesaffre et Compagnie whose address is 41 rue Etienne Marcel, 75009 Paris;
identification reference “SCPro-1” and registration number “I-3856” deposited at the National Collection of Cultures of Microorganisms (CNCM) (25, rue du Docteur Roux, 75724 Paris cedex 15) in France, on Oct. 17, 2007 by Lesaffre et Compagnie whose address is 41 rue Etienne Marcel, 75009 PARIS.

1-14. (canceled)

15. A method for preventing and/or treating vaginal candidiasis in a subject comprising administering to the subject in need thereof a therapeutically effective amount of Limosilactobacillus fermentum strain chosen among the strain deposited with the CNCM on Nov. 17, 2021 under number I-5777 or under number I-5778.

16. The method according to claim 15, wherein the vaginal candidiasis is due to fungi selected from the group consisting of Candida albicans, Candida tropicalis, Candida pseudotropicalis, Candida krusei, Candida glabrata, and mixtures thereof.

17. The method according to claim 15, wherein the strain is used in association with the Saccharomyces cerevisiae strain deposited with the CNCM on Oct. 17, 2007 under number I-3856 and/or with the yeast obtained by culturing said Saccharomyces cerevisiae strain.

18. The method according to claim 17, wherein the yeast obtained by culturing the Saccharomyces cerevisiae strain is in the form of dry yeast or fresh yeast, and preferably in the form of dry yeast.

19. The method according to claim 18, wherein the dry yeast is in the form of active dry yeast or instant dry yeast.

20. The method according to claim 17, wherein the yeast is in the form of inactive yeast.

21. The method according to claim 17, wherein the yeast is in the form of a yeast derivative, said derivative being chosen among the cell wall of said yeast, the cell wall glucans of said yeast, the cell wall mannoproteins of said yeast, or mixtures thereof.

22. A method for preventing and/or treating vaginal candidiasis in a subject comprising administering to the subject in need thereof a therapeutically effective amount bacteria obtained by culturing the Limosilactobacillus fermentum strain chosen among the strain deposited with the CNCM on Nov. 17, 2021 under number I-5777 or under number I-5778.

23. The method according to claim 22, wherein the bacteria is presented in in frozen form or in dry form, preferably in dry form, and more preferably still in freeze-dried dry form.

24. The method according to claim 22, wherein the vaginal candidiasis is due to fungi selected from the group consisting of Candida albicans, Candida tropicalis, Candida pseudotropicalis, Candida krusei, Candida glabrata, and mixtures thereof.

25. The method according to claim 22, wherein the bacteria is used in association with the Saccharomyces cerevisiae strain deposited with the CNCM on Oct. 17, 2007 under number I-3856 and/or with the yeast obtained by culturing said Saccharomyces cerevisiae strain.

26. The method according to claim 25, wherein the yeast obtained by culturing the Saccharomyces cerevisiae strain is in the form of dry yeast or fresh yeast, and preferably in the form of dry yeast.

27. The method according to claim 26, wherein the dry yeast is in the form of active dry yeast or instant dry yeast.

28. The method according to claim 25, wherein the yeast is in the form of inactive yeast.

29. The method according to claim 25, wherein the yeast is in the form of a yeast derivative, said derivative being chosen among the cell wall of said yeast, the cell wall glucans of said yeast, the cell wall mannoproteins of said yeast, or mixtures thereof.

30. A composition comprising:
a Limosilactobacillus fermentum strain chosen among the strain deposited with the CNCM on Nov. 17, 2021 under number I-5777 or under number I-5778, and/or a bacteria obtained by culturing said Limosilactobacillus fermentum strain,
the Saccharomyces cerevisiae strain deposited with the CNCM on Oct. 17, 2007 under number I-3856 and/or the yeast obtained by culturing said Saccharomyces cerevisiae strain,
possibly an excipient,
for use in the prevention and/or treatment of vaginal candidiasis.

31. The composition according to claim 30, wherein the composition is a pharmaceutical composition, a food composition, or a food supplement.

32. The composition according to claim 30, wherein it is in a form suitable for oral or vaginal administration, preferably oral.

33. The composition according to claim 30, further comprising an active ingredient selected from the group consisting of vitamin K, vitamin B9, glutathione, S-adenosyl-L-methionine (SAM-e), chondroitin sulfate, and mixtures thereof.

34. The composition according to claim 30, wherein:
Limosilactobacillus fermentum bacterial strain or bacteria I-5777 and/or I-5778 is in dry form, preferably freeze-dried, and is present in a quantity ranging from 1×10⁷ to 1×10¹⁰ CFU, preferably from 2×10⁸ to 2×10⁹ CFU, and
yeast Sc I-3856 is in dry form, and is present in a quantity ranging from 1×10⁷ to 1×10¹¹ CFU, preferably from 1×10⁹ to 1×10¹⁰ CFU, and even more preferably from 2.5×10⁹ to 5×10⁹ CFU.
